SEQUENCE LISTING (7 sequences)

SEQ ID NO: 1 - protein, 6 aa, Neisseria gonorrhoeae
GSGGGG

SEQ ID NO: 2 - protein, 225 aa, Neisseria gonorrhoeae
MTFFKPSTVVLTASALALSGCVADPVTGQQSPNKSAMYGLGGAAVCGIVGALTHSGKGARNSALACGAIGAGVGGYMDYQEQRLRQNLAGTQIEIQRQGNQIRLVMPESVTFATGSAALGGSAQYALNTAAQTLVQYPDTTLTINGHTDNTGSDAVNNPLSQHRAQAVAYYLQTRGVAASRLTVYGYGSHMPVASNATVEGRAQNRRVEILINPDQRAVNAARHM

SEQ ID NO: 3 - protein, 185 aa, Neisseria gonorrhoeae
MPSEALQTAFRGNIRRSFTMIRLTRAFAAALIGLCCTTGAHADTFQKIGFINTERIYLESKQARNIQKTLDGEFSARQDELQKLQREGLDLERQLAGGKLKDAKKAQAEEKWRGLVEAFRKKQAQFEEDYNLRRNEEFASLQQNANRVIVKIAKQEGYDVILQDVIYVNTQYDVTDSVIKEMNAR

SEQ ID NO: 4 - protein, 272 aa, Neisseria gonorrhoeae
MNTIFKISALTLSAALALSACGKKEAAPASASEPAAASAAQGDTSSIGSTMQQASYAMGVDIGRSLKQMKEQGAEIDLKVFTDAMQAVYDGKEIKMTEEQAQEVMMKFLQEQQAKAVEKHKADAKANKEKGEAFLKENAAKDGVKTTASGLQYKITKQGEGKQPTKDDIVTVEYEGRLIDGTVFDSSKANGGPATFPLSQVIPGWTEGVRLLKEGGEATFYIPSNLAYREQGAGEKIGPNATLVFDVKLVKIGAPENAPAKQPDQVDIKKVN

SEQ ID NO: 5 - protein, 288 aa, Neisseria gonorrhoeae
MILASLVRYYRRLATETDETGNPKVPSYGFSEEKIGWILVLDKEGRLKTVVPNLTADKKPQPKLMSVPRPEKRTSGIKPNFLWDKTAYALGVEANKNKAEAKEKPFTPSEKTFEAFKQYHLDLLQNSEDEGLQALCRFLQNWQPAHFAAENLPAEMLDSNTAFSLEKPTALIHKREAAQTLWAGCLKSDEALESLCLISGDTAPIARLHPAIKGVFGGQSSGGSIISFNKEAFSSFGKEQGANAPVSEQSAFAYTTALNYLLRRENNHCLTIGDASTVFWAEADDIVD

SEQ ID NO: 6 - protein, 550 aa, Neisseria gonorrhoeae
MVAYAFLFLFVTAAVLLIVRSHYRWTYFFASALFVFLAGGMLMLTAQWQRALNFASVWFVVLILFHRLKIHYYKQPLLISDFLLIADWRNWETLFHYKEAVIGMAGLLALAGYAVFGWSGADSLGMPWRWAGAVLFAAAFVSVRHFSKHPGAVKTWLDSLPDDGRDVFLNLPMSCRAVFFQVPVFEGDGEAFARQMPSETRPYGMSDEKPDIVVTLMESTLDPHCFDFAAAKIPDLKMFGRQEDTVFSSPLRVHTFGGATWKSEFAFLAGVPSTDFGALASGVFYSVVPHLQTGFVRNLREHGYFCVALSPFTKGNYNAKAAYDHFGFNLMFQPQDLGYPAPMGKNLWHISSEEMMQYARMILEKRHPDLENVRQPMFVYVLTMKEHGPYRTDTDNVFDLDAPDLNAKTVSALNDYIGRIADLDKAVESFDRYLHERGKPFVFGYFGDHQVPFEGVSVRKKWDYAQPDYVTQFAVRSNIAGGFVQRQDFLDLAFAGGVLMEAAGLEAKDGFMRANMAMRGLCGGGLEDCPNRELVGNYRNYLYDVLKIAR

SEQ ID NO: 7 - protein, 1468 aa, Neisseria gonorrhoeae
MKTTDKRTTETHRKAPKTGRIRFSPAYLAICLSFGILPQARAGHTYFGINYQYYRDFAENKGKFAVGAKDIEVYNKKGELVGKSMTKAPMIDFSVVSRNGVAALAGDQYIVSVAHNGGYNNVDFGAEGSNPDQHRFSYQIVKRNNYKAGTNGHPYGGDYHMPRLHKFVTDAEPVEMTSYMDGWKYADLNKYPDRVRIGAGRQYWRSDEDEPNNRESSYHIASAYSWLVGGNTFAQNGSGGGTVNLGSEKIKHSPYGFLPTGGSFGDSGSPMFIYDAQKQKWLINGVLQTGNPYIGKSNGFQLVRKDWFYDEIFAGDTHSVFYEPHQNGKYFFNDNNNGAGKIDAKHKHYSLPYRLKTRTVQLFNVSLSETAREPVYHAAGGVNSYRPRLNNGENISFIDKGKGELILTSNINQGAGGLYFEGNFTVSPKNNETWQGAGVHISDGSTVTWKVNGVANDRLSKIGKGTLLVQAKGENQGSVSVGDGKVILDQQADDQGKKQAFSEIGLVSGRGTVQLNADNQFNPDKLYFGFRGGRLDLNGHSLSFHRIQNTDEGAMIVNHNQDKESTVTITGNKDITTTGNNNNLDSKKEIAYNGWFGEKDATKTNGGLNLNYPPEEADRTLLLSGGTNLNGNITQTNGKLFFSGRPTPHAYNHLGSGWSKMEGIPQGEIVWDNDWIDRTFKAENFHIQGGQAVVSRNVAKVEGDWHLSNHAQAVFGVAPHQSHTICTRSDWTGLTSCTEKTITDDKVIASLSKTDVRGNVSLADHAHLNLTGLATFNGNLVQAETRTIRLRANATQNGNLSLVGNAQATFNQATLNGNTSASDNASFNLSNNAVQNGSLTLSDNAKANVSHSALNGNVSLADKAVFHFENSRFTGKISGGKDTALHLKDSEWTLPSGTELGNLNLDNATITLNSAYRHDAAGAQTGSAADAPRRRSRRSLLSVTPPTSAESRFNTLTVNGKLNGQGTFRFMSELFGYRSGKLKLAESSEGTYTLAVNNTGNEPVSLEQLTVVEGKDNTPLSENLNFTLQNEHVDAGAWRYQLIRKDGEFRLHNPVKEQELSDKLGKAGETEAALTAKQAQLAAKQQAEKDNAQSLDALIAAGRNATEKAESVAEPARQAGGENAGIMQAEEEKKRVQADKDTALAKQREAETRPATTAFPRARRARRDLPQPQPQPQPQPQRDLISRYANSGLSEFSATLNSVFAVQDELDRVFAEDRRNAVWTSGIRDTKHYRSQDFRAYRQQTDLRQIGMQKNLGSGRVGILFSHNRTGNTFDDGIGNSARLAHGAVFGQYGIGRFDIGISAGAGFSSGSLSDGIRGKIRRRVLHYGIQARYRAGFGGFGIEPHIGATRYFVQKADYRYENVNIATPGLAFNRYRAGIKADYSFKPAQHISITPYLSLSYTDAASGKVRTRVNTAVLAQDFGKTRSAEWGVNAEIKGFTLSLHAAAAKGPQLEAQHSAGIKLGYRW

(19) United States
(12) Patent Application Publication
(10) Pub. No.: US 2008/0166370 A1
(43) Pub. Date: Jul. 10, 2008

(54) Immunogenic Compositions Comprising Multiple Gonococcal Antigens

(75) Inventors: Laura Serino, Montiliano (IT); Rosanna Leuzzi, Siena (IT); Maria Rita Fontana, Siena (IT); Elisabetta Monaci, Castiglione D'Orcia (IT); Mariagrazia Pizza, Siena (IT)

Correspondence Address: NOVARTIS VACCINES AND DIAGNOSTICS INC., INTELLECTUAL PROPERTY R338, P.O. BOX 8097, Emeryville, CA, 94662-8097, US

(73) Assignee: CHIRON SRL, SIENA (IT)

(21) Appl. No.: 10/561,235

(22) PCT Filed: Jun. 25, 2004
(86) PCT No.: PCT/IB2004/002421

§ 371 Date: Nov. 20, 2006

(30) Foreign Application Priority Data
Jun. 26, 2003 (GB) 0315021.6

(51) Int. Cl. A61K 39/02; C07H 21/04; C12N 9/16

(52) U.S. Cl. 424/200.1; 424/234.1; 530/350; 536/23.7; 435/252.3; 435/196

(57) ABSTRACT

N. gonorrhoeae is a bacterial pathogen which causes diseases including gonorrhoea, urethritis, cervicitis and pelvic inflammatory disease. In addition, like other inflammatory SYDs, infection is believed to enhance HIV transmission. Within the many proteins of the gonococcal *enome, six have been found to be particularly suitable for immunisation purposes, particularly', when used in combinations. The invention therefore provides a composition comprising two or more of the following antigens: (1) OmpA; (2) OmpH; (3) PPIase; (4) ngs41; (5) ngsl 17; and (6)', App.

All documents cited herein are incorporated by reference in their entirety.

### TECHNICAL FIELD

This invention is in the fields of immunology and vaccinology. In particular, it relates to antigens derived from Neisseria gonorrhoeae (gonococcus) and their use in immunisation.

### BACKGROUND ART

N. gonorrhoeae is a bacterial pathogen which causes diseases including gonorrhoea, urethritis, cervicitis and pelvic inflammatory disease. In addition, like other inflammatory STDs, infection is believed to enhance HIV transmission.

N. gonorrhoeae is related to N. meningitidis (meningococcus). Sequence data are now available for serogroup B of meningococcus {e.g. refs. 1 to 6} and also for serogroup A {7}. It is a further object of the invention to provide proteins and nucleic acid useful in distinguishing between gonococcus and meningococcus and, in particular, between gonococcus and serogroup B meningococcus.

Various gonococcal antigens have been described {e.g. ref. 8}, but there is currently no effective vaccine against N. gonorrhoeae infection. It is an object of the invention to provide materials useful in vaccine development.

Vaccines against pathogens such as hepatitis B virus, diphtheria and tetanus typically contain a single protein antigen (e.g. the HBV surface antigen, or a tetanus toxoid). In contrast, acellular whooping cough vaccines typically have at least three B. pertussis proteins, and the Prevenar™ pneumococcal vaccine contains seven separate conjugated saccharide antigens. Other vaccines such as cellular pertussis vaccines, the measles vaccine, the inactivated polio vaccine (IPV) and meningococcal OMV vaccines are by their very nature complex mixtures of a large number of antigens. Whether protection against can be elicited by a single antigen, a small number of defined antigens, or a complex mixture of undefined antigens, therefore depends on the pathogen in question.

Gonococcal infection provokes a massive inflammatory response in genitourinary mucosae and a consequent infiltration of mononuclear phagocytes, including a significant number of macrophages, in subepithelial tissues. While the primary interaction of N. gonorrhoeae with human phagocytes is mediated by pili and opacity outer membrane protein (Opa), very little is known on the fate of gonococci after the internalization, although it is likely that entry and survival of gonococci into resident macrophages play an important role in the persistent phases of inflammation as well as in the spread of microorganisms.

It is an object of the invention to provide further and improved compositions for providing immunity against gonococcal disease and/or infection. It is a further objection to provide compositions for use in minimising macrophage invasion by gonococcus. The compositions are based on a combination of two or more gonococcal antigens.

### DISCLOSURE OF THE INVENTION

Within the many proteins of the gonococcal genome, six have been found to be particularly suitable for immunisation purposes, particularly when used in combinations. The invention therefore provides a composition comprising two or more of the following antigens: (1) OmpA; (2) OmpH; (3) PPIase; (4) ngs41; (5) ngsl 17; and (6) App. These are referred to herein as the ‘six basic antigens’.

The composition may comprise three or more, four or more, five or more, or all six of the six basic antigens. Preferred compositions comprise: (1) OmpA & OmpH; (2) OmpA & PPIase; (3) OmpA & ngs41; (4) OmpA & ngsl 17; (5) OmpA & App; (6) OmpH & PPIase; (7) OmpH & ngs41; (8) OmpH & ngsl 17; (9) OmpH & App; (10) PPIase & ngs41; (11) PPIase & ngsl 17; (12) PPIase & App; (13) ngs41 & ngsl 17; (14) ngs41 & App; and (15) ngsl 17 & App.

(1) OmpA protein

The ‘OmpA’ protein has been disclosed as SEQ ID ^{S}: 25 & 26 in reference 8 (SEQ ID : 2 herein).

Preferred OmpA proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID : 2; and/or (b) which is a fragment of at least n consecutive amino acids of SEQ ID 2, wherein n is 7 or more (e.g. 8, 10, 12, 14, 16, 18, 20, 25, 30, 20 35, 40, 50, 60, 70, 80, 90, 100, 150, 200, 250 or more). These OmpA proteins include variants (e.g. allelic variants, homologs, orthologs, paralogs, mutants, etc.) of SEQ ID : 2. Preferred fragments of (b) comprise an epitope from SEQ ID : 2. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25, 45 or more) from the N-terminus of SEQ ID : 2. Other fragments omit one or more domains of the protein (e.g. omission of a signal peptide, of a cytoplasmic domain, of a transmembrane domain, or of an extracellular domain). The transmembrane domain of OmpA (numbered relative to SEQ ID : 1) is at around residues 36-52, and the Gram negative signal peptide is around residues 1-23.

The protein may be lipidated (e.g. by a N-acyl diglyceride), and may thus have a N-terminal cysteine.

### (2) OmpH Protein

The sequence of ‘OmpH’ protein in gonococcal strain FA1090 is SEQ ID : 3 herein (see also SEQ ID ^{S}: 6055 & 6056 of reference 8).

Preferred OmpH proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID : 3; and/or (b) which is a fragment of at least n consecutive amino acids of SEQ ID 3, wherein n is 7 or more (e.g. 8, 10, 12, 14, 16, 18, 20, 25, 30, 35, 40, 50, 60, 70, 80, 90, 100, 150, 200, 250 or more). These OmpH proteins include variants (e.g. allelic variants, homologs, orthologs, paralogs, mutants, etc.) of SEQ ID : 3. Preferred fragments of (b) comprise an epitope from SEQ ID : 3. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more; preferably at least 19) from the N-terminus of SEQ ID : 3. Other fragments omit one or more domains of the protein (e.g. omission of a signal peptide, of a cytoplasmic domain, of a transmembrane domain e.g. residues 20-36 of SEQ ID :3, or of an extracellular domain).

Residues 74-129 may form a coiled-coil domain, and so the OmpH protein may be present in the form of an oligomer e.g. a dimer, trimer, tetramer, etc.

(3) Peptidyl-prolyl cis/trans Isomerase (PPIase) Protein

The ‘PPIase’ protein has been disclosed as part of SEQ ID ^{S}: 1033 & 1034 in reference 8 (SEQ ID : 4 herein).

Preferred PPIase proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID : 4; and/or (b) which is a fragment of at least n consecutive amino acids of SEQ ID 4, wherein n is 7 or more (e.g. 8, 10, 12, 14, 16, 18, 20, 25, 30, 35, 40, 50, 60, 70, 80, 90, 100, 150, 200, 250 or more). These PPIase proteins include variants (e.g. allelic variants, homologs, orthologs, paralogs, mutants, etc.) of SEQ ID : 4. Preferred fragments of (b) comprise an epitope from SEQ ID : 4. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID : 4. Other fragments omit one or more domains of the protein (e.g. omission of a signal peptide, of a cytoplasmic domain, of a transmembrane domain, or of an extracellular domain).

The protein may be lipidated (e.g. by a N-acyl diglyceride), and may thus have a N-terminal cysteine.

The protein may be present in the form of an oligomer e.g. a dimer.

### (4) Ngs41 Protein

The ‘Ngs41’ protein has been disclosed as SEQ ID ^{S}: 81 & 82 in reference 8 (SEQ ID : 5 herein).

Preferred Ngs41 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID : 5; and/or (b) which is a fragment of at least n consecutive amino acids of SEQ ID 5, wherein n is 7 or more (e.g. 8, 10, 12, 14, 16, 18, 20, 25, 30, 35, 40, 50, 60, 70, 80, 90, 100, 150, 200, 250 or more). These Ngs41 proteins include variants (e.g. allelic variants, homologs, orthologs, paralogs, mutants, etc.) of SEQ ID : 5. Preferred fragments of (b) comprise an epitope from SEQ ID : 5. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID : 5. Other fragments omit one or more domains of the protein (e.g. omission of a signal peptide, of a cytoplasmic domain, of a transmembrane domain, or of an extracellular domain).

### (5) Ngsl 17 Protein

The ‘Ngs117’ protein has been disclosed as SEQ ID ^{S}: 233 & 234 in reference 8 (SEQ ID : 6 herein).

Preferred Ngs117 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID : 6; and/or (b) which is a fragment of at least n consecutive amino acids of SEQ ID 6, wherein n is 7 or more (e.g. 8, 10, 12, 14, 16, 18, 20, 25, 30, 35, 40, 50, 60, 70, 80, 90, 100, 150, 200, 250 or more). These Ngs117 proteins include variants (e.g. allelic variants, homologs, orthologs, paralogs, mutants, etc.) of SEQ ID : 6. Preferred fragments of (b) comprise an epitope from SEQ ID : 6. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID : 6. Other fragments omit one or more domains of the protein (e.g. omission of a signal peptide, of a cytoplasmic domain, of a transmembrane domain, or of an extracellular domain).

### (6) App

The gonococcal ‘App’ protein has been disclosed as SEQ ID ^{S}: 653 & 654 in reference 1, and as SEQ ID ^{S}: 1087 & 1088 in reference 8 (SEQ ID : 7 herein). It is related to the meningococcal adhesion penetration protein (App) disclosed in reference 9.

Preferred App proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID : 7; and/or (b) which is a fragment of at least n consecutive amino acids of SEQ ID 7, wherein n is 7 or more (e.g. 8, 10, 12, 14, 16, 18, 20, 25, 30, 35, 40, 50, 60, 70, 80, 90, 100, 150, 200, 250 or more). These App proteins include variants (e.g. allelic variants, homologs, orthologs, paralogs, mutants, etc.) of SEQ ID : 7. Preferred fragments of (b) comprise an epitope from SEQ ID : 7. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID : 7. Other fragments omit one or more domains of the protein (e.g. omission of a signal peptide, of a cytoplasmic domain, of a transmembrane domain, or of an extracellular domain). The App protein is subject to autoproteolysis, and so a proteolytic fragment of SEQ ID : 7 may be used.

### Fusion Proteins

The six basic antigens may be present in the composition as six separate polypeptides, but it is preferred that at least two (i.e. 2, 3, 4, 5 or 6) of the antigens are expressed as a single polypeptide chain (a ‘hybrid’ polypeptide) e.g. such that the six antigens form fewer than six polypeptides. Hybrid polypeptides offer two principal advantages: first, a polypeptide that may be unstable or poorly expressed on its own can be assisted by adding a suitable hybrid partner that overcomes the problem; second, commercial manufacture is simplified as only one expression and purification need be employed in order to produce two polypeptides which are both antigenically useful.

A hybrid polypeptide included in a composition of the invention may comprise two or more (i.e. 2, 3, 4, 5, 6) of the six basic antigens. Hybrids consisting of two or three of the six basic antigens are preferred.

Within the combination of six basic antigens, an antigen may be present in more than one hybrid polypeptide and/or as a non-hybrid polypeptide. It is preferred, however, that an antigen is present either as a hybrid or as a non-hybrid, but not as both.

Two-antigen hybrids for use in the invention comprise: (1) OmpA & OmpH; (2) OmpA & PPIase; (3) OmpA & ngs41; (4) OmpA & ngs117; (5) OmpA & App; (6) OmpH & PPIase; (7) OmpH & ngs41; (8) OmpH & ngs117; (9) OmpH & App; (10) PPIase & ngs41; (11) PPIase & ngs117; (12) PPIase & App; (13) ngs41 & ngs117; (14) ngs41 & App; and (15) ngs117 & App.

Hybrid polypeptides can be represented by the formula NH₂—A—{—X—L—}ₙ—B—COOH, wherein: X is an amino acid sequence of one of the six basic antigens as defined above; L is an optional linker amino acid sequence; A is an optional N-terminal amino acid sequence; B is an optional C-terminal amino acid sequence; and n is 2, 3, 4, 5, 6, 7, 8, 9, 10, 11, 12, 13 14 or 15.

If a —X— moiety has a leader peptide sequence in its wild-type form, this may be included or omitted in the hybrid protein. In some embodiments, the leader peptides will be deleted except for that of the —X— moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety —A—.

For each n instances of {—X—L—}, linker amino acid sequence —L— may be present or absent. For instance, when n=2 the hybrid may be NH₂—X₁—L₁—X₂—L₂—COOH, NH₂—X₁—X₂—COOH, NH₂—X₁—L₁—X₂—COOH, NH₂—X₁—X₂—L₂—COOH, etc. Linker amino acid sequence(s) —L— will typically be short (e.g. 20 or fewer amino acids i.e. 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art. A useful linker is GSGGGG (SEQ ID 1), with the Gly—Ser dipeptide being formed from a BamHI restriction site, thus aiding cloning and manipulation, and the (Gly)₄ tetrapeptide being a typical poly-glycine linker.

—A— is an optional N-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. Hisₙ where n=3, 4, 5, 6, 7, 8, 9, 10 or more). Other suitable N-terminal amino acid sequences will be apparent to those skilled in the art. If XI lacks its own N-terminus methionine, —A— is preferably an oligopeptide (e.g. with 1, 2, 3, 4, 5, 6, 7 or 8 amino acids) which provides a N-terminus methionine.

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminal amino acid sequences will be apparent to those skilled in the art.

Most preferably, n is 2 or 3.

The invention also provides nucleic acid encoding hybrid polypeptides of the invention. Furthermore, the invention provides nucleic acid which can hybridise to this nucleic acid, preferably under “high stringency” conditions (e.g. 65° C. in a 0.1×SSC, 0.5% SDS solution). Polypeptides of the invention can be prepared by various means (e.g. recombinant expression, purification from cell culture, chemical synthesis, etc.) and in various forms (e.g. native, fusions, non-glycosylated, lapidated, etc.). They are preferably prepared in substantially pure form (i.e. substantially free from other neisserial or host cell proteins).

Nucleic acid according to the invention can be prepared in many ways (e.g. by chemical synthesis, from genomic or cDNA libraries, from the organism itself, etc.) and can take various forms (e.g. single stranded, double stranded, vectors, probes, etc.). They are preferably prepared in substantially pure form (i.e. substantially free from other neisserial or host cell nucleic acids).

The term “nucleic acid” includes DNA and RNA, and also their analogues, such as those containing modified backbones (e.g. phosphorothioates, etc.), and also peptide nucleic acids (PNA), etc. The invention includes nucleic acid comprising sequences complementary to those described above (e.g. for antisense or probing purposes).

The invention also provides a process for producing a polypeptide of the invention, comprising the step of culturing a host cell transformed with nucleic acid of the invention under conditions which induce polypeptide expression.

The invention provides a process for producing a polypeptide of the invention, comprising the step of synthesising at least part of the polypeptide by chemical means.

The invention provides a process for producing nucleic acid of the invention, comprising the step of amplifying nucleic acid using a primer-based amplification method (e.g. PCR).

The invention provides a process for producing nucleic acid of the invention, comprising the step of synthesising at least part of the nucleic acid by chemical means.

### Strains

Preferred polypeptides of the invention comprise an amino acid sequence found in gonococcal strain FA1090.

Where hybrid polypeptides are used, the individual antigens within the hybrid (i.e. individual —X— moieties) may be from one or more strains. Where n=2, for instance, X₂ may be from the same strain as X₁ or from a different strain. Where n=3, the strains might be (i) X₁=X₂=X₃ (ii) X₁=X₂≠X₃ (iii) X₁≠X₂=X₃ (iv) X₁≠X₂≠X₃ or (v) X₁=X₃≠X₂, etc.

### Heterologous Host

Whilst expression of the polypeptides of the invention may take place in gonococcus, the invention preferably utilises a heterologous host. The heterologous host may be prokaryotic (e.g. a bacterium) or eukaryotic. It is preferably E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc.

### Immunogenic Compositions and Medicaments

Compositions of the invention are preferably immunogenic compositions, and are more preferably vaccine compositions. The pH of the composition is preferably between 6 and 8, preferably about 7. The pH may be maintained by the use of a buffer. The composition may be sterile and/or pyrogen-free. The composition may be isotonic with respect to humans.

Vaccines according to the invention may either be prophylactic (i.e. to prevent infection) or therapeutic (i.e. to treat infection), but will typically be prophylactic.

The invention also provides a composition of the invention for use as a medicament. The medicament is preferably able to raise an immune response in a mammal (i.e. it is an immunogenic composition) and is more preferably a vaccine.

The invention also provides the use of two or more (e.g. 3, 4, 5, 6) of the six basic antigens in the manufacture of a medicament for raising an immune response in a mammal. The medicament is preferably a vaccine.

The invention also provides a method for raising an immune response in a mammal comprising the step of administering an effective amount of a composition of the invention. The immune response is preferably protective and preferably involves antibodies and/or cell-mediated immunity. The method may raise a booster response.

The mammal is preferably a human. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

These uses and methods are preferably for the prevention and/or treatment of a disease caused by a gonococcus (e.g. gonorrhoea, urethritis, cervicitis and pelvic inflammatory disease, etc.).

One way of checking efficacy of therapeutic treatment involves monitoring gonococcal infection after administration of the composition of the invention. One way of checking efficacy of prophylactic treatment involves monitoring immune responses against the six basic antigens after administration of the composition.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral (e.g. tablet, spray), vaginal, topical, transdermal {e.g. see ref. 10} or transcutaneous {e.g. see refs. 11 & 12}, intranasal {e.g. see ref. 13}, ocular, aural, pulmonary or other mucosal administration.

The invention may be used to elicit systemic and/or mucosal immunity.

Dosage treatment can be a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc.

Gonococcal infections affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

### Further Components of the Composition

The composition of the invention will typically, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. A thorough discussion of pharmaceutically acceptable excipients is available in reference 14.

Vaccines of the invention may be administered in conjunction with other immunoregulatory agents. In particular, compositions will usually include an adjuvant. Preferred further adjuvants include, but are not limited to: (A) aluminium salts, including hydroxides (e.g. oxyhydroxides), phosphates (e.g. hydroxyphosphates, orthophosphates), sulphates, etc. {e.g. see chapters 8 & 9 of ref. 15}), or mixtures of different aluminium compounds, with the compounds taking any suitable form (e.g. gel, crystalline, amorphous, etc.), and with adsorption being preferred; (B) MF59 (5% Squalene, 0.5% Tween 80, and 0.5% Span 85, formulated into submicron particles using a microfluidizer) {see Chapter 10 of 15; see also ref. 16); (C) liposomes (see Chapters 13 and 14 of ref. 15); (D) ISCOMs (see Chapter 23 of ref. 15), which may be devoid of additional detergent {17}; (E) SAF, containing 10% Squalane, 0.4% Tween 80, 5% pluronic-block polymer L121, and thr-MDP, either microfluidized into a submicron emulsion or vortexed to generate a larger particle size emulsion {see Chapter 12 of ref. 15}; (F) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (G) saponin adjuvants, such as QuilA or QS21 {see Chapter 22 of ref. 15}, also known as Stimulon™ {18}; (H) chitosan {e.g. 19}; (I) complete Freund's adjuvant (CFA) and incomplete Freund's adjuvant (IFA); (J) cytokines, such as interleukins (e.g. IL-1, IL-2, IL-4, IL-5, IL-6, IL-7, IL-12, etc.), interferons (e.g. interferon-γ), macrophage colony stimulating factor, tumor necrosis factor, etc. (see Chapters 27 & 28 of ref. 15}; (K) monophosphoryl lipid A (MPL) or 3-O-deacylated MPL (3 dMPL) {e.g. chapter 21 of ref. 15}; (L) combinations of 3 dMPL with, for example, QS21 and/or oil-in-water emulsions {20}; (M) a polyoxyethylene ether or a polyoxyethylene ester {21}; (N) a polyoxyethylene sorbitan ester surfactant in combination with an octoxynol {22} or a polyoxyethylene alkyl ether or ester surfactant in combination with at least one additional non-ionic surfactant such as an octoxynol {23}; (N) a particle of metal salt {24}; (O) a saponin and an oil-in-water emulsion {25}; (P) a saponin (e.g. QS21)+3 dMPL+IL-12 (optionally +a sterol) {26}; (Q) E. coli heat-labile enterotoxin (“LT”), or detoxified mutants thereof, such as the K63 or R72 mutants {e.g. Chapter 5 of ref. 27}; (R) cholera toxin (“CT”), or detoxified mutants thereof {e.g. Chapter 5 of ref. 27}; (S) double-stranded RNA; (T) microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) being preferred, optionally treated to have a negatively-charged surface (e.g. with SDS) or a positively-charged surface (e.g. with a cationic detergent, such as CTAB); (U) oligonucleotides comprising CpG motifs i.e. containing at least one CG dinucleotide; (V) monophosphoryl lipid A mimics, such as aminoalkyl glucosaminide phosphate derivatives e.g. RC-529 {28}; (W) polyphosphazene (PCPP); (X) a bioadhesive {29} such as esterified hyaluronic acid microspheres {30} or a mucoadhesive selected from the group consisting of cross-linked derivatives of poly(acrylic acid), polyvinyl alcohol, polyvinyl pyrollidone, polysaccharides and carboxymethylcellulose; or (Y) other substances that act as immunostimulating agents to enhance the effectiveness of the composition {e.g. see Chapter 7 of ref. 15}. Aluminium salts and MF59 are preferred adjuvants for parenteral immunisation. Mutant toxins are preferred mucosal adjuvants.

Muramyl peptides include N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine MTP-PE), etc. The composition may include an antibiotic.

### Further Antigens

The composition contains six basic antigens. It may also include further antigens, although it can contain no gonococcal protein antigens other than the six basic antigens. Further antigens for inclusion may be, for example:
a saccharide antigen from N. meningitidis serogroup A, C, W135 and/or Y, such as the oligosaccharide disclosed in ref. 31 from serogroup C {see also ref. 32} or the oligosaccharides of ref. 33.
antigens from Helicobacter pylon such as CagA {34 to 37}, VacA {38, 39}, NAP {40, 41, 42}, HopX {e.g. 43}, HopY {e.g. 43} and/or urease.
a saccharide antigen from Streptococcus pneumoniae {e.g. 44, 45, 46}.
a protein antigen from Streptococcus pneumoniae {e.g. 47}.
an antigen from hepatitis A virus, such as inactivated virus {e.g. 48, 49}.
an antigen from hepatitis B virus, such as the surface and/or core antigens {e.g. 49, 50}.
an antigen from hepatitis C virus {e.g. 51}.
a diphtheria antigen, such as a diphtheria toxoid {e.g. chapter 3 of ref. 52} e.g. the CRM₁₉₇ mutant {e.g. 53}.
a tetanus antigen, such as a tetanus toxoid {e.g. chapter 4 of ref. 52}.
an antigen from Bordetella pertussis, such as pertussis holotoxin (PT) and filamentous haemagglutinin (FHA) from B. pertussis, optionally also in combination with pertactin and/or agglutinogens 2 and 3 {e.g. refs. 54 & 55}; whole-cell pertussis antigen may also be used.
a saccharide antigen from Haemophilus influenzae B {e.g. 32}.
polio antigen(s) {e.g. 56, 57} such as OPV or, preferably, IPV.
a protein antigen from N. meningitidis serogroup B {e.g. refs. 1-6 & 58-63}
an outer-membrane vesicle (OMV) preparation from N. meningitidis serogroup B, such as those disclosed in refs. 64, 65, 66, 67, etc.
an antigen from Chlamydia trachomatis {e.g. 68}.
an antigen from Chlamydia pneumoniae {e.g. refs. 69 to 75}.
an antigen from Porphyromonas gingivalis {e.g. 76}.
an antigen from Treponema pallidum.
rabies antigen(s) {e.g. 77} such as lyophilised inactivated virus {e.g. 78, RabAvert™}.
measles, mumps and/or rubella antigens {e.g. chapters 9, 10 & 11 of ref. 52}.
influenza antigen(s) {e.g. chapter 19 of ref. 52}, such as the haemagglutinin and/or neuraminidase surface proteins.
antigen(s) from a paramyxovirus such as respiratory syncytial virus (RSV {79, 80}) and/or parainfluenza virus (PIV3 {81}).
an antigen from Moraxella catarrhalis {e.g. 82}.
an antigen from Streptococcus pyogenes (group A streptococcus) {e.g. 83, 84, 85}.
an antigen from Streptococcus agalactiae (group B streptococcus) {e.g. 86}.
an antigen from Staphylococcus aureus {e.g. 87}.
an antigen from Bacillus anthracis {e.g. 88, 89, 90}.
a papillomavirus antigen e.g. from any HPV type.
a herpes simplex virus antigen e.g. from HSV-1 or HSV-2.
an antigen from a virus in the flaviviridae family (genus flavivirus), such as from yellow fever virus, Japanese encephalitis virus, four serotypes of Dengue viruses, tick-borne encephalitis virus, West Nile virus.
an antigen from a HIV e.g. a HIV-1 or HIV-2.
an antigen from a rotavirus.
a pestivirus antigen, such as from classical porcine fever virus, bovine viral diarrhoea virus, and/or border disease virus.
a parvovirus antigen e.g. from parvovirus B19.
a coronavirus antigen e.g. from the SARS coronoavirus.
a prion protein (e.g. the CJD prion protein)
an amyloid protein, such as a beta peptide {91}
a cancer antigen, such as those listed in Table 1 of ref. 92 or in tables 3 & 4 of ref. 93.

The composition may comprise one or more of these further antigens. The composition may include at least one further bacterial antigen and/or at least one further viral antigen. It is preferred that combinations of antigens should be based on shared characteristics e.g. antigens associated with respiratory diseases, antigens associated with enteric diseases, antigens associated with sexually-transmitted diseases, etc.

Where a saccharide or carbohydrate antigen is used, it is preferably conjugated to a carrier protein in order to enhance immunogenicity {e.g. refs. 94 to 103}. Preferred carrier proteins are bacterial toxins or toxoids, such as diphtheria or tetanus toxoids. The CRM₁₉₇ diphtheria toxoid is particularly preferred {104}. Other carrier polypeptides include the N. meningitidis outer membrane protein {105}, synthetic peptides {106, 107}, heat shock proteins {108, 109}, pertussis proteins {110, 111}, protein D from H. influenzae {112}, cytokines {113}, lymphokines {113}, hormones {113}, growth factors {113}, toxin A or B from C. difficile {114}, iron-uptake proteins {115}, etc. Where a mixture comprises capsular saccharides from both serogroups A and C, it may be preferred that the ratio (w/w) of MenA saccharide: MenC saccharide is greater than 1 (e.g. 2:1, 3:1, 4:1, 5:1, 10:1 or higher). Different saccharides can be conjugated to the same or different type of carrier protein. Any suitable conjugation reaction can be used, with any suitable linker where necessary.

Toxic protein antigens may be detoxified where necessary e.g. detoxification of pertussis toxin by chemical and/or genetic means {55}.

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens in the composition will typically be present at a concentration of at least 1 μg/ml each. In general, the concentration of any given antigen will be sufficient to elicit an immune response against that antigen.

As an alternative to using protein antigens in the composition of the invention, nucleic acid encoding the antigen may be used {e.g. refs. 116 to 124}. Protein components of the compositions of the invention may thus be replaced by nucleic acid (preferably DNA e.g. in the form of a plasmid) that encodes the protein.

### Knockout Mutants

The invention provides gonococcal knockout mutants, wherein a gene encoding one or more of the six basic antigens has been knocked out. The mutant is preferably an isogenic knockout mutant.

The knockout mutant does not detectably express the knocked-out antigen.

### Definitions

The term “comprising” encompasses “including” as well as “consisting” e.g. a composition “comprising” X may consist exclusively of X or may include something additional e.g. X+Y.

The term “about” in relation to a numerical value x means, for example, x±10%.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of reference 125. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in reference 126.

### BRIEF DESCRIPTION OF THE DRAWINGS

FIG. 1 shows western blotting using anti-OmpA serum. FIG. 2 shows similar data for anti-OmpH serum. FIG. 3 shows anti-OmpH western blot data for a variety of clinical isolates.

FIGS. 4 and 6 show expression of PPIase in extracts of cell culture over time. FIG. 5 shows anti-PPIase western blot data for a variety of clinical isolates.

FIG. 7 shows a western blot of whole cells using anti-App serum. App is seen in the intact cells of two strains (lanes 1 & 3) but not in the isogenic knockout mutants (lanes 2 & 4). FIG. 8 is a western blot showing App expression over time. FIGS. 9 and 10 show FACS analysis of App expression.

FIG. 11 shows non-reducing SDS-PAGE analysis of purified recombinant PPIase.

FIG. 12 shows inhibition of PPIase activity by rapamycin at nanomolar concentrations.

FIG. 13 shows (A) total cell-associated bacteria and (B) total intracellular bacteria in an assay on human macrophages. Grey bars are with a knockout strain; white bars are with a wild-type strain.

FIG. 14 shows immunofluorescence microscopy of human macrophages, stained using an antiserum against gonococcal OMV as primary antibody. Macrophages were incubated with either (A) wild-type F62 or (B) Δ576. FIG. 15 shows similar experiments with the ME180 cell line.

FIG. 16 shows SDS-PAGE analysis of OmpA expression. Lanes: (1) empty plasmid; (2) 1.5 hours after IPTG induction; (3) 3 hours after IPTG induction.

FIG. 17 shows FACS analysis of gonococcal OmpA in E. coli. Anti-OmpA polyclonal mouse serum (diluted 1:500) was the primary antibody, and FITC-conjugated anti-mouse IgG (diluted 1:100) as secondary antibody.

FIG. 18 is a growth curve of wild-type and OmpA-knockout gonococcus.

FIG. 19 shows immunofluorescence of (A) PBS-treated and (B) OmpA-incubated cells.

FIG. 20 shows immunofluorescence of cells infected with (A) wild-type and (B) OmpA-knockouts.

FIG. 21 shows microscopy of monolayers of mouse macrophage cells incubated with (A) wild-type and (B) OmpA-knockout bacteria.

FIG. 22 shows western blot analysis of OmpA in various gonococcal strains.

FIG. 23 shows results of C4bp binding to OmpA at different OmpA concentrations. No binding is seen to the negative control protein (Ctl−).

FIG. 24 shows ELISA analysis for the interaction of C4bp with OmpA. The top curve shows OmpA, and the bottom three curves are negative control proteins.

### MODES FOR CARRYING OUT THE INVENTION

### The Six Basic Antigens

The six antigens OmpA, OmpH, PPIase, ngs41, ngs117 and App were individually expressed in E. coli and purified. Antibodies against the six proteins were made in mice, and the antibodies were used for western blots against gonococcus F62, to detect cell surface expression.

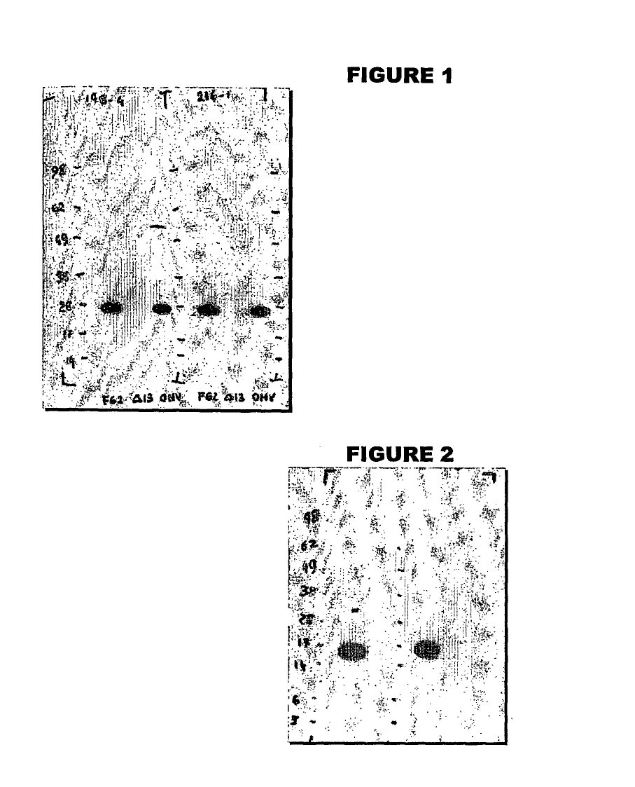

The OmpA protein could be seen in gonococcus using the anti-OmpA serum (FIG. 1, lanes 1 & 4). It could also be seen in OMVs prepared from gonococcus (lanes 3 & 6). In isogenic deletion mutants, however, no immunoreactive band could be seen (lanes 2 & 5)

The OmpH protein was detected in gonococcus by the anti-OmpH sera (FIG. 2, lanes 1 & 3). In isogenic knockout mutants of gonococcus, however, no immunoreactive band was visible (FIG. 2, lanes 2 & 4). Expression of OmpH across various clinical isolates was also tested by western blot. As shown in FIG. 3, immunoreactive bands were seen in isolates from Baltimore USA (top left), from the UK (bottom left) and from Korea (top right).

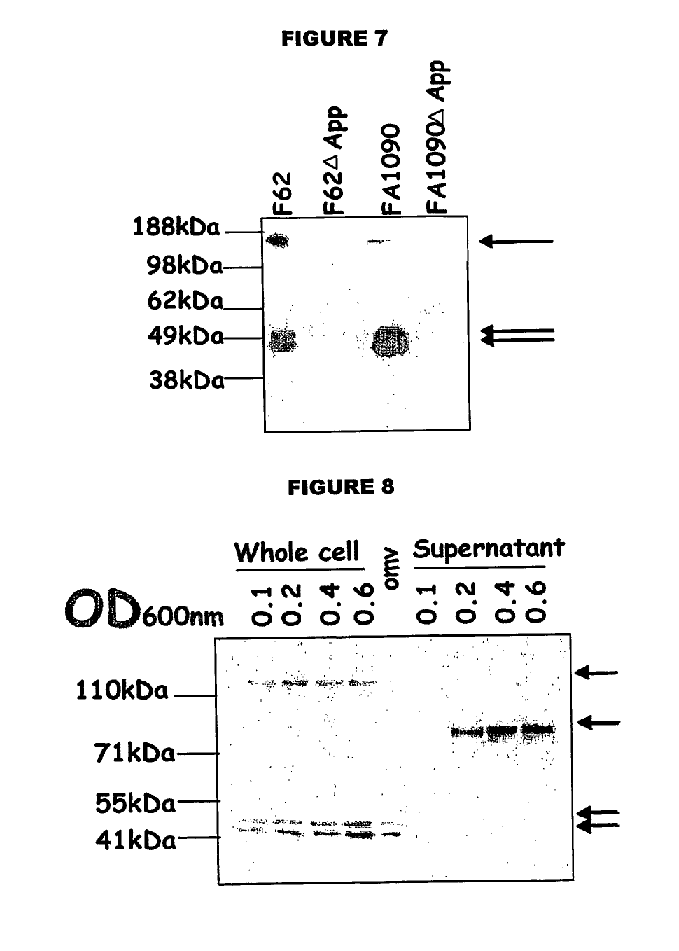

Autotransporters, such as App, are synthesised as large precursor proteins comprising at least three functional domains: the N-terminal leader sequence, the passenger domain, and the C-terminal domain (β-domain). The leader sequence mediates the export of the protein in to the periplasm, the β-domain inserts into the outer membrane and allows the export of the passenger domain. Once at the bacterial surface, the passenger domain can be cleaved and released in the environment. The expression data for gonococcal App was consistent with this model—full-length protein was seen on the cell surface of F62 and FA1090 strains by western blot (FIG. 7, showing full-length ˜160 kDa protein and also cleavage products; see also FIG. 8, lanes 14) and by FACS (FIG. 9), was seen by western blot on the surface of OMVs prepared from log-phase cells (FIG. 8, lane 5), was found by western blot to be processed and secreted in the culture supernatant (FIG. 8, lanes 6-9), but no protein was detected when using isogenic knockouts either by western blot (FIG. 7) or by FACS (FIG. 9). In addition, a C3 binding assay showed that App is able to elicit antibodies which activate the complement cascade (FIG. 10).

### Adhesion Studies

The role of the six basic antigens in gonococcal adhesion was studied using knockout strains. The ability of wild-type and knockout strains to bind to and then invade ME-180 (epithelial-like human cells from cervical carcinoma) or Hec1B (epithelial-like human cells from endometrial adenocarcinoma) cells was compared.

Adhesion assays were performed using the epithelial cells seeded in 96-well tissue-culture plates and grown in Medium 199 with the addition of 10% FCS, until confluency. Gonococci grown on GC agar were suspended in Dulbecco's complete phosphate-buffered saline (PBSB) and used to infect cell monolayers at 200-100 bacteria/cell. At the end of a 3-hour incubation at 37° C. in 5% CO₂ (v/v), total colony-forming units (cfu) were estimated after addition of 1% saponin to the wells. Adhesiveness was quantified by determining the ratio of cell-associated cfu/total cfu present in the assay.

For invasion experiments, intracellular bacteria were recovered after treatment for 2 hours with gentamicin (200 μg/ml), to kill extracellular bacteria. Results were presented as ratio of the adhesiveness of the tested strain to that of the high-adhesive control.

OmpH knockouts showed a 7-fold reduction in adhesion and a 12-fold reduction in invasion. Ngs13 knockouts showed a 2-fold reduction in adhesion and a 5-fold reduction in invasion. PPIase knockouts showed a 30-fold reduction in adhesion and a similar reduction in invasion. App knockouts showed a 2-fold reduction in adhesion and a 5-fold reduction in invasion.

### PPIase

SEQ ID : 4 shows 43% sequence identity to macrophage infectivity potentiator (MIP) from Legionella pneumophila, which is a PPIase that promotes the early step of intracellular infectivity.

Peptidyl-prolyl-cis/trans isomerase activity catalyses the slow cis/trans isomerisation of prolyl peptide bonds involved in the proline-mediated folding of proteins. PPIases belong to the prokaryotic and eukaryotic family of FK506-binding proteins (FKBP), inhibited by the macrolide antibiotic FK506 and rapamycin.

The PPIase activity of the gonococcal protein has been confirmed by an in vitro assay on a purified recombinant protein comprising SEQ ID : 4, expressed in E. coli with a C-terminus histidine tag. A chymotrypsin-coupled assay was tested on two substrates, and k_{cat}/K_{M} was determined for both:

| Substrate | K_{cat}/Kₘ (M⁻¹ sec⁻¹) |
|---|---|
| Succinyl-ala-phe-pro-phe-nitroanilide | 4.1 × 10⁵ |
| Succinyl-ala-ala-pro-phe-nitroanilide | 5.74 × 10⁴ |

The PPIase activity is inhibited by rapamycin at nanomolar concentration (FIG. 12).

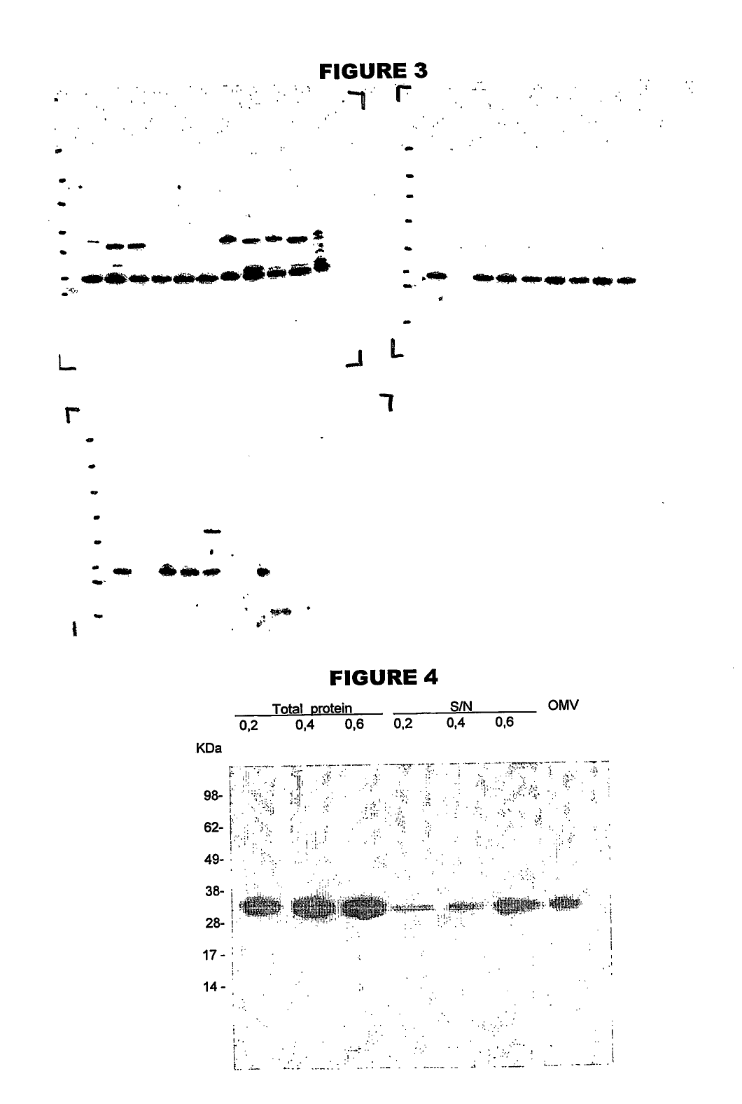

In the F62 strain, PPIase protein is detected in the total cell extracts as time progresses. The protein is secreted in the culture supernatant during growth when OD₆₀₀ₙₘ is 0.2, 0.4 and 0.6 (FIG. 4) and is also present in the outer membrane vesicles (OMV) indicating a surface-localization.

PPIase is present in total extracts obtained from all clinical isolates analysed (10 from Baltimore, 7 from Korea and 4 from England). The positive and negative control are the strain F62 and the relative isogenic mutant Δ576 (FIG. 5).

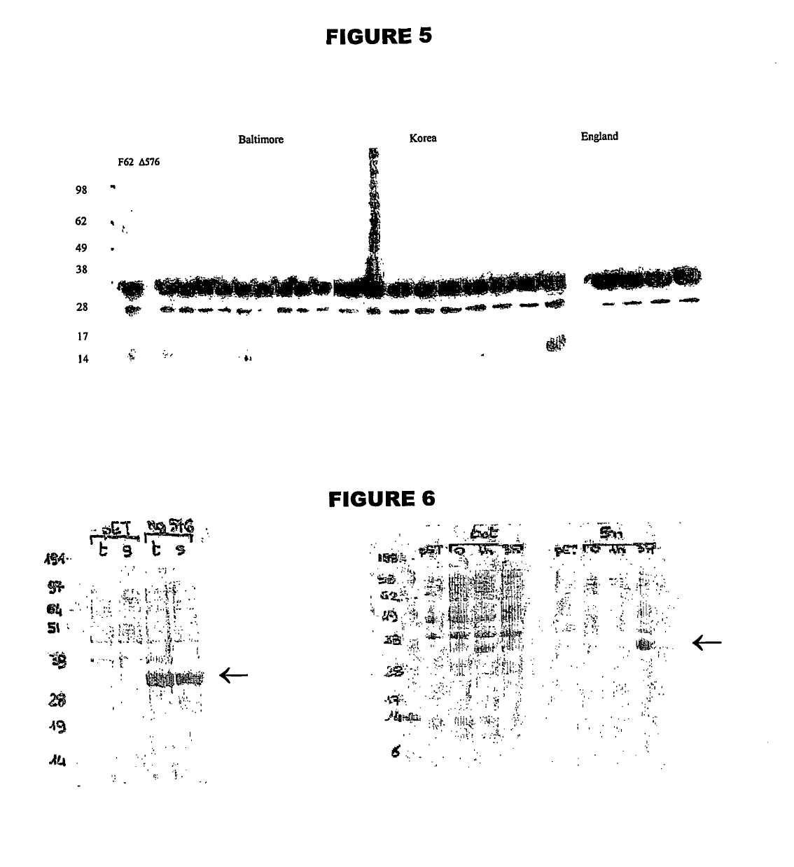

The native form of PPIase was cloned in the expression vector pET under the T7 promoter and expressed in E. coli BL21 (DE3) strain. After 1 hour of IPTG induction (FIG. 6, left panel) the protein is detected in total extract (t) and in soluble fraction (s). The protein is progressively secreted in the culture supernatant (FIG. 6, Sn in right panel).

The ability of gonococci to survive intracellularly in the RAW264 cell line was assessed for wild-type and for the Δ576 knockout. The number of intracellular bacteria was determined after 30 min, 1 hour and 3 hours of infection followed by gentamicin treatment. In the knockout strain there is a reduction of 3-10 fold of intracellular survival.

Adhesion and invasion assays using cell lines showed that the Δ576 knockout mutant was less able to adhere to and also to invade Hec1b human endometrial cells and ME180 human cervical cells. The reduced level of adherent bacteria in ME180 cells was confirmed by immunofluorescence microscopy, as shown in FIG. 15.

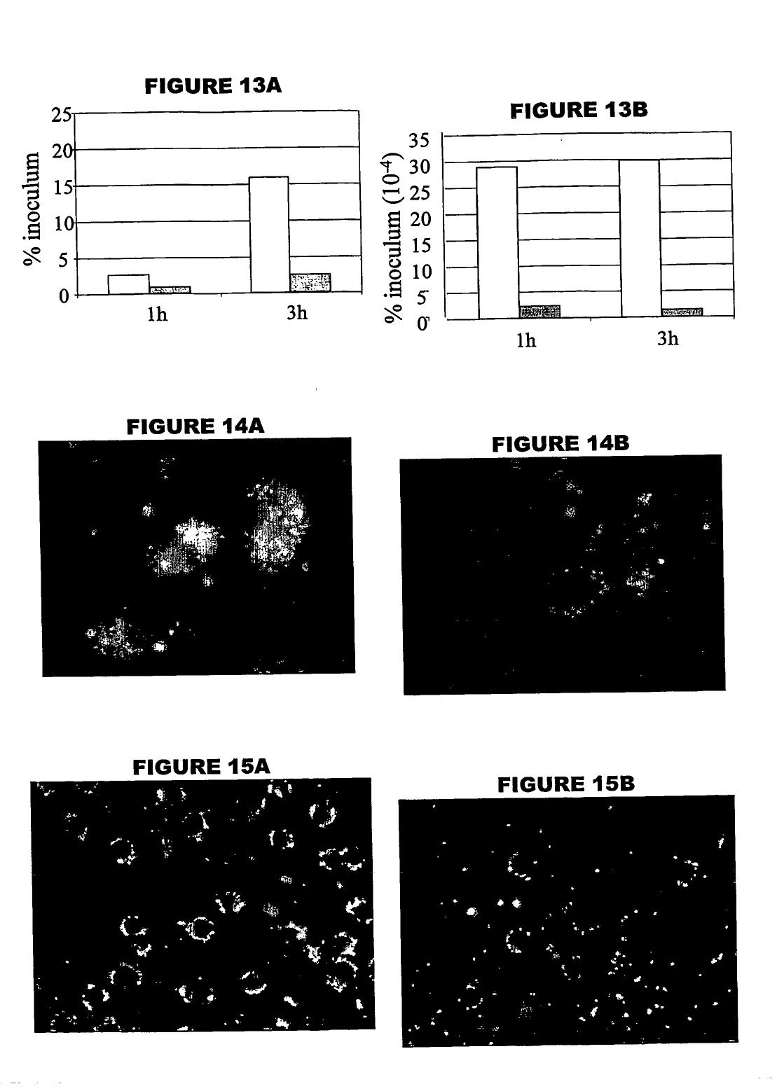

Similar studies were performed using human macrophages, derived from monocytes isolated from human blood. These macrophages were incubated for either 1 hour or 3 hours with either F62 strain gonococcus or with the isogenic mutant Δ576. Cell-associated bacteria were then counted. The number of intracellular bacteria were also counted after gentamicin treatment. The results of this analysis are shown in FIG. 13. FIG. 13A shows that the total cell-associated bacteria were 5-fold less with the Δ576 strain, and FIG. 13B shows that total intracellular bacteria were 20-fold less with the knockout strain. The reduced level of intracellular bacteria was confirmed by immunofluorescence microscopy. Macrophages were infected with F62 and Δ576 strains, and the results are shown in FIG. 14.

Further studies were performed using macrophage-differentiated U937 human cell line. The cell lines, differentiated in macrophages by PMA treatment, were infected for 1 or 3 hours as before. The total cell-associated bacteria were 4-5 fold less in the Δ576 strain relative to wild-type, and the number of intracellular bacteria, determined after gentamicin treatment, was about 6-fold less.

PPIase may not be involved in the primary interaction with phagocytes, but may play an important role in the phases subsequent to macrophage-mediated internalization. Intracellular gonococci of the knockout strain are considerably more sensitive to macrophage-mediated killing and undergo a time-dependent decrease. PPIase plays a role in the persistence of N. gonorrhoeae in macrophages.

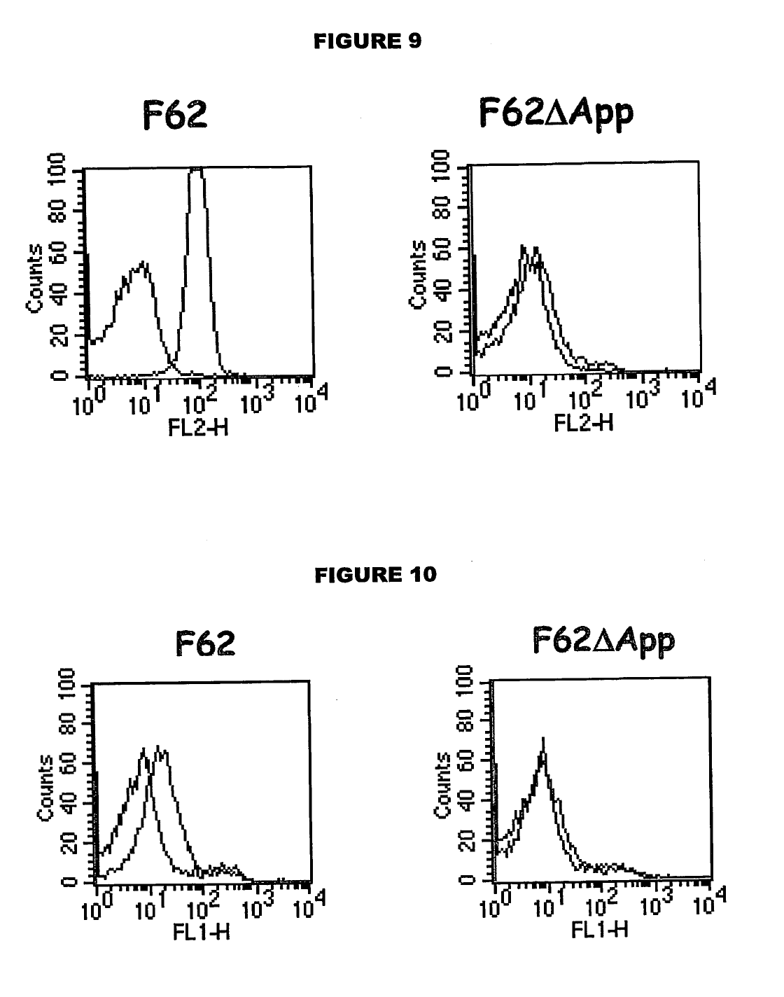

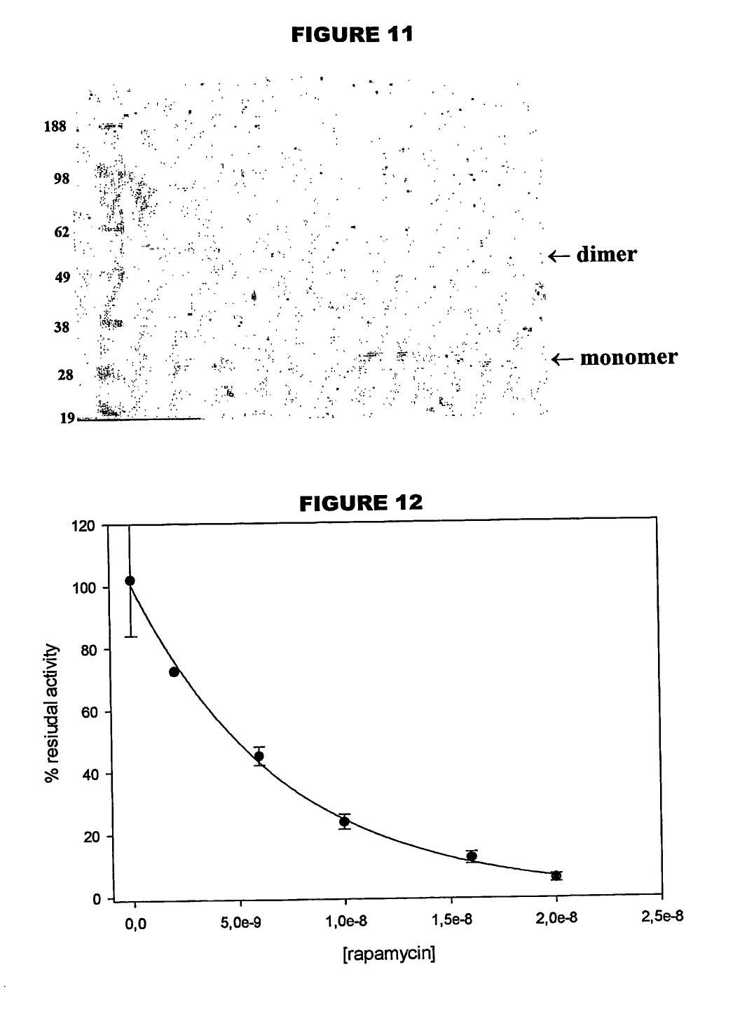

Purified recombinant PPIase was analysed by gel filtration and was seen to form dimers in solution. The dimer can also be seen in non-reducing SDS-PAGE (FIG. 11).

### OmpA

Within OmpA proteins, gonococcal OmpA is most closely related to Vitreoscilla spp, with about 61% sequence identity (76% similarity). Identity to OmpA from other species (including E. coli, Salmonella, Yersinia and Pseudomonas) ranges from 40% to 46%. There is no homologous ompA gene in N. meningitidis, wherein the gene is absent and replaced by a truncated transposase, although the flanking genes are well conserved between the two species.

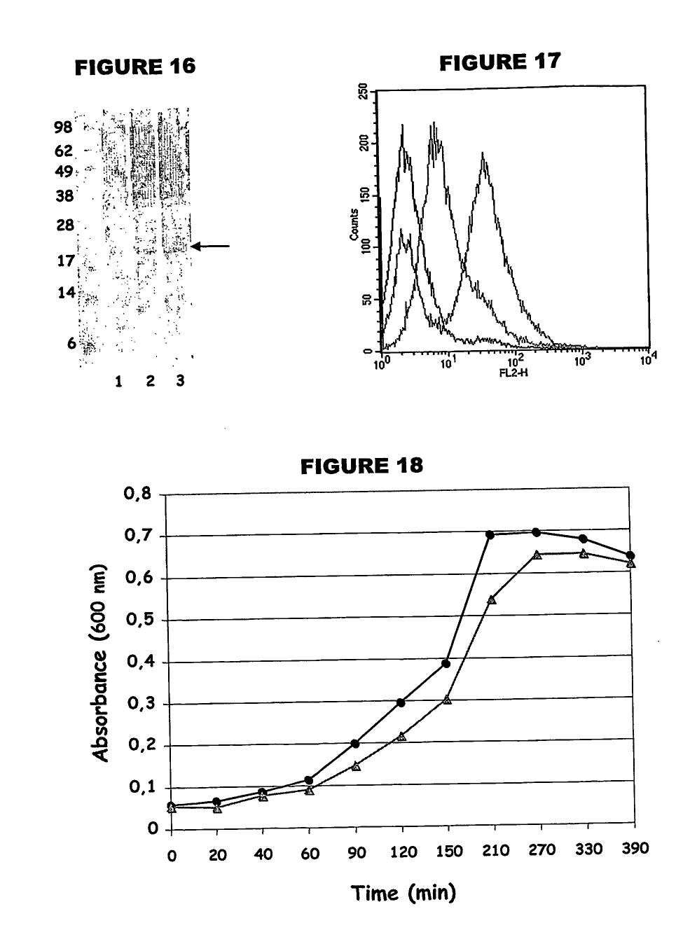

The gonococcal ompA gene was cloned under the control of the T7 promoter in the expression vector pET21b, to give pET-OmpA-His. This plasmid was introduced into E. coli BL21 (DE3) and the protein was produced with a C-terminal His-tag. The protein was used to raise antibodies in mice. As shown in FIG. 16, expression of native OmpA in E. coli was detected by SDS-PAGE analysis after IPTG induction (0, 1.5 and 3 h) in the total cell extracts of BL21/pET-OmpA (lanes 2 and 3). FACS analysis of E. coli hyper-expressing gonococcal OmpA confirms cell-surface location (FIG. 17).

An ompA isogenic mutant strain was constructed, by cloning ˜600 bp of the upstream and downstream flanking regions in the vector pBluescript and replacing the entire gene by the erythromycin resistance cassette. Growth of the knockout mutant was not affected in GC liquid medium over 8 hours, compared to wild-type F62 (FIG. 18).

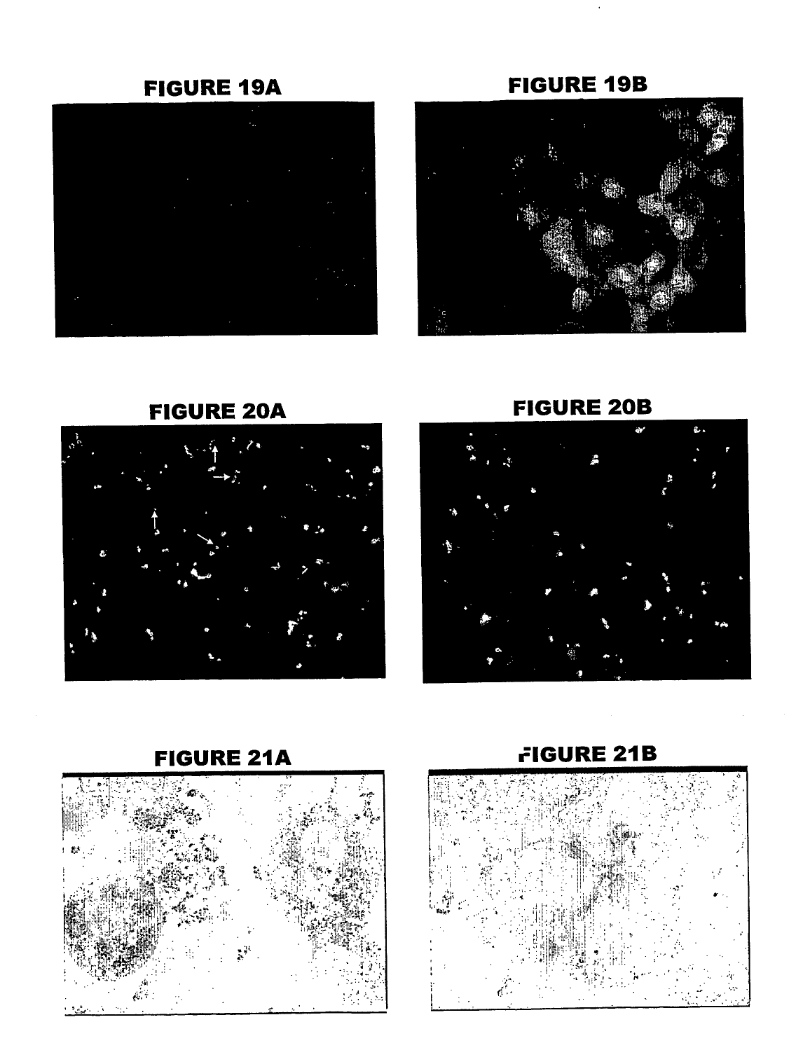

Immunofluorescence microscopy showed that purified OmpA protein binds to ME-180 human cervical epithelial cells. Monolayers of the ME-180 cells were treated with PBS (control) or with 1 mg/ml purified OmpA, labeled using polyclonal mouse antiserum against recombinant Ng OmpA-His, followed by anti-mouse Alexa Fluor 488 conjugated antibodies. The results are in FIG. 19.

Adherence and invasion of N. gonorrhoeae strain F62 and the isogenic knockout mutant ΔOmpA to human endometrial (Hec-1B) and cervical carcinoma cells (ME-180) was investigated. Bacteria were allowed to adhere to cell monolayers for 3 hours. Cell-associated and intracellular bacteria were both decreased for both cell types. Thus OmpA protein was shown to play a significant role in the adhesion and invasion process into human cervical carcinoma and endometrial cells.

Immuno double fluorescence staining of extracellular (green) and intracellular (red) bacteria was performed on monolayers of ME-180 cells infected with wild type F62 and ΔOmpA mutant. Non-adherent bacteria were removed by washings. Cells were fixed and incubated with primary polyclonal antibody. The cells were then incubated with Alexa Fluor 488 secondary antibodies. After permeabilization with Triton X-100, cells were incubated with the primary antibody to label internalized bacteria, followed by incubation with Alexa Fluor 568 secondary antibodies. Wild-type F62 bound to significant numbers on the cell monolayers and some bacteria were observed inside the cells (white arrows; FIG. 20A). In contrast, very few ΔOmpA bacteria either bound to or entered the cells (FIG. 20B).

Similar experiments were performed with a macrophage cell line. Monolayers of mouse macrophage cells RAW264 were infected with N. gonorrhoeae strain F62 and the isogenic ΔOmpA knockout mutant. Bacteria were incubated with the cells for 3 hours and then extracellular bacteria were removed by several washes. After fixing the cells, Giemsa staining was used. Very few ΔOmpA strains entered and survived inside the macrophages (FIG. 21). Thus OmpA is implicated in entry and intracellular survival into macrophages.

It is known that some antigens contribute to serum resistance by binding to complement regulatory protein C4b binding protein (C4bp), leading to a decrease in serum killing by complement attack. N. gonorrhoeae OmpA was found to bind to C4bp. Dot blot ligand overlay analysis showed C4bp (10 mg/ml) binding to purified OmpA protein at different concentrations (from 0.1 mg to 4 mg), as shown in FIG. 23. In further experiments, purified OmpA and three negative control proteins were immobilized in the wells of a microtiter plate at different concentrations (from 0.15 mg to 20 mg). The wells were blocked with 5% skimmed milk in PBS and then incubated with 10% NHS as source of C4bp. After washing, bound C4bp was detected (FIG. 24).

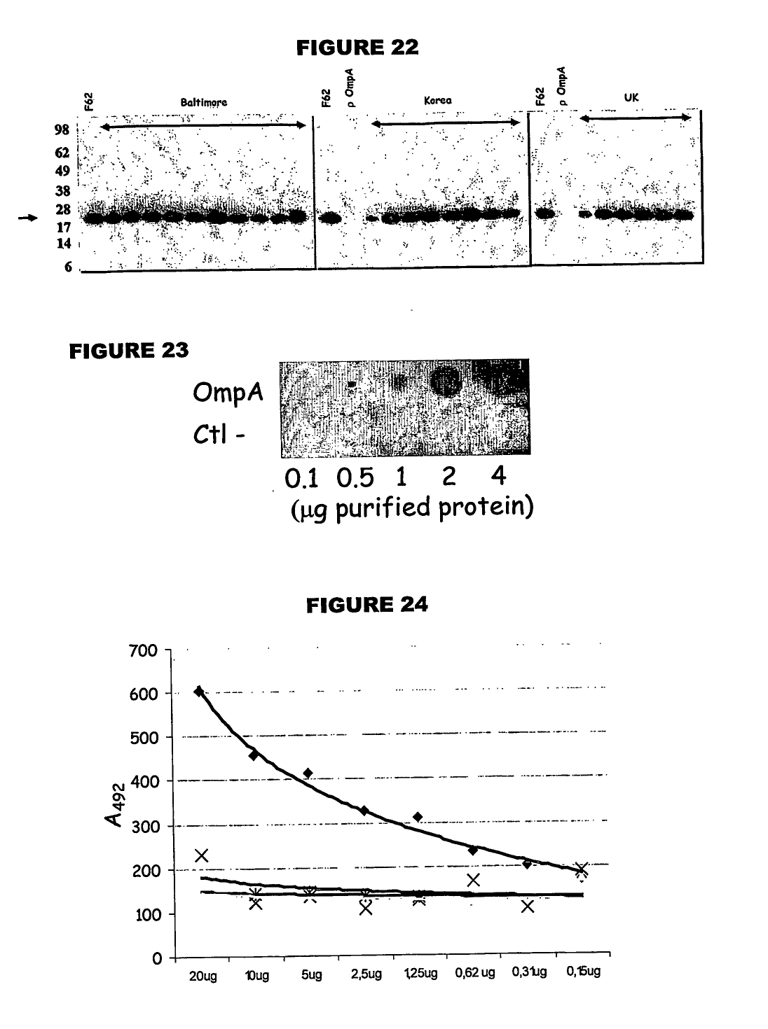

A panel of clinical strains obtained from different geographical areas were investigated, and OmpA expression was seen in all isolates at comparable levels. Western blots were labeled using polyclonal mouse antiserum against recombinant Ng OmpA-His, as primary antibody. As positive and negative controls, wild type F62 strain and the isogenic knockout mutant ΔOmpA were used (FIG. 22).

OmpA has been expressed in E. coli and confirmed to be surface located both in N. gonorrhoeae and E. coli, using FACS analysis and Western blotting. The protein is well conserved in gonococcus and its expression has been observed in all clinical isolates analyzed from different geographical areas. The purified OmpA protein is able to bind to human epithelial cells. An isogenic knockout ΔOmpA mutant shows reduced levels of adhesion and invasion into human endometrial and adenocarcinama cells (Hec-1B and ME-180). The expression of OmpA appears to be also required for intracellular survival of N. gonorrhoeae into human macrophages. OmpA is able to bind the complement regulatory protein C4bp, suggesting a role of this protein in contributing to the serum resistance of the strain by evasion of the complement attack.

These observations suggest that OmpA represents an important factor involved in both the interaction between N. gonorrhoeae and the human host and establishing the infection process.

### Combinations

After expression and purification, the six antigens were combined in pairs, triples, quadruples, etc. The efficacy of the combined antigens was tested in a mouse model of N. gonorrhoeae infection and was compared to the efficacy of the antigens alone, and also against adjuvant-only controls. The antigens (single and combined) were administered to the mice in combination with various adjuvants.

It will be understood that the invention has been described by way of example only and modifications may be made whilst remaining within the scope and spirit of the invention.

### REFERENCES (THE CONTENTS OF WHICH ARE HEREBY INCORPORATED BY REFERENCE)

{1} WO99/24578.
{2} WO99/36544.
{3} WO99/57280.
{4} WO00/22430.
{5} Tettelin et al. (2000) Science 287: 1809-1815.
{6} Pizza et al. (2000) Science 287: 1816-1820.
{7} Parkhill et al. (2000) Nature 404: 502-506
{8} WO02/079243.
{9} Hadi et al. (2001) Mol. Microbiol. 41: 611-623.
{10} WO99/27961.
{11} WO02/074244.
{12} WO02/064162.
{13} WO03/028760.
{14} Gennaro (2000) Remington: The Science and Practice of Pharmacy. 20th ed., ISBN: 0683306472.
{15} Vaccine design: the subunit and adjuvant approach (1995) Powell & Newman. ISBN 0-306-44867-X.
{16} WO90/14837.
{17} WO00/07621.
{18} WO00/62800.
{19} WO99/27960.
{20} European patent applications 0835318, 0735898 and 0761231.
{21} WO99/52549.
{22} WO01/21207.
{23} WO01/21152.
{24} WO00/23105.
{25} WO99/11241.
{26} WO98/57659.
{27} Del Giudice et al. (1998) Molecular Aspects of Medicine, vol. 19, number 1.
{28} Johnson et al. (1999) Bioorg Med Chem Lett 9: 2273-2278.
{29} WO00/50078.
{30} Singh et al. (2001) J. Cont. Rele. 70: 267-276.
{31} Costantino et al. (1992) Vaccine 10: 691-698.
{32} Costantino et al. (1999) Vaccine 17: 1251-1263.
{33} WO03/007985.
{34} Covacci & Rappuoli (2000) J. Exp. Med. 19: 587-592.
{35} WO93/18150.
{36} Covacci et al. (1993) Proc. Natl. Acad. Sci. USA 90: 5791-5795.
{37} Tummuru et al. (1994) Infect. Immun. 61: 1799-1809.
{38} Marchetti et al. (1998) Vaccine 16: 33-37.
{39} Telford et al. (1994) J. Exp. Med. 179: 1653-1658.
{40} Evans et al. (1995) Gene 153: 123-127.
{41} WO96/01272 & WO96/01273, especially SEQ ID :6.
{42} WO97/25429.
{43} WO98/04702.
{44} Watson (2000) Pediatr Infect Dis J 19: 331-332.
{45} Rubin (2000) Pediatr Clin North Am 47: 269-285, v.
{46} Jedrzejas (2001) Microbiol Mol Biol Rev 65: 187-207.
{47} WO02/077021.
{48} Bell (2000) Pediatr Infect Dis J 19: 1187-1188.
{49} Iwarson (1995)APMIS 103: 321-326.
{50} Gerlich et al. (1990) Vaccine 8 Suppl: S63-68 & 79-80.
{51} Hsu et al. (1999) Clin Liver Dis 3:901-915.
{52} Vaccines (1988) eds. Plotkin & Mortimer. ISBN 0-7216-1946-0.
{53} Del Guidice et al. (1998) Molecular Aspects of Medicine 19: 1-70.
{54} Gustafsson et al. (1996) N. Engl. J. Med. 334: 349-355.
{55} Rappuoli et al. (1991) TIBTECH 9: 232-238.
{56} Sutter et al. (2000) Pediatr Clin North Am 47: 287-308.
{57} Zimmerman & Spann (1999) Am Fain Physician 59: 113-118, 125-126.
{58} WO00/66791.
{59} WO03/020756.
{60} WO01/64920.
{61} WO01/64922.
{62} UK patent application 0227346.4 (particularly international application claiming priority therefrom).
{63} UK patent applications 0223741.0, 0305831.0 & 0309115.4 (particularly international application claiming priorities therefrom).
{64} Bjune et al. (1991) Lancet 338 (8775): 1093-96
{65} WO01/52885.
{66} Fukasawa et al. (1999) Vaccine 17: 2951-2958.
{67} Rosenqvist et al. (1998) Dev. Biol. Stand. 92: 323-333.
{68} WO99/28475.
{69} WO02/02606.
{70} Kalman et al. (1999) Nature Genetics 21: 385-389.
{71} Read et al. (2000) Nucleic Acids Res 28: 1397-406.
{72} Shirai et al. (2000) J. Infect. Dis. 181 (Suppl 3): S524-S527.
{73} WO99/27105.
{74} WO00/27994.
{75} WO00/37494.
{76} Ross et al. (2001) Vaccine 19: 4135-4142.
{77} Dreesen (1997) Vaccine 15 Suppl: S2-6.
{78} MMWR Morb Mortal Wkly Rep 1998 Jan 16; 47(1): 12, 19.
{79} Anderson (2000) Vaccine 19 Suppl 1: S59-65.
{80} Kahn (2000) Curr Opin Pediatr 12: 257-262.
{81} Crowe (1995) Vaccine 13: 415-421.
{82} McMichael (2000) Vaccine 19 Suppl 1: S101-107.
{83} WO02/34771.
{84} Dale (1999) Infect Dis Clin North Am 13: 227-43, viii.
{85} Ferretti et al. (2001) PNAS USA 98: 4658-4663.
{86} WO02/34771.
{87} Kuroda et al. (2001) Lancet 357 (9264): 1225-1240; see also pages 1218-1219.
{88} J Toxicol Clin Toxicol (2001) 39: 85-100.
{89} Demicheli et al. (1998) Vaccine 16: 880-884.
{90} Stepanov et al. (1996) J Biotechnol 44: 155-160.
{91} Ingram (2001) Trends Neurosci 24: 305-307.
{92} Rosenberg (2001) Nature 411: 380-384.
{93} Moingeon (2001) Vaccine 19: 1305-1326.
{94} Ramsay et al. (2001) Lancet 357 (9251): 195-196.
{95} Lindberg (1999) Vaccine 17 Suppl 2: S28-36.
{96} Buttery & Moxon (2000) J R Coll Physicians Lond 34: 163-168.
{97} Ahmad & Chapnick (1999) Infect Dis Clin North Am 13: 113-133, vii.
{98} Goldblatt (1998) J. Med. Microbiol. 47: 563-567.
{99} European patent 0 477 508.
{100} U.S. Pat. No. 5,306,492.
{101} WO98/42721.
{102} Conjugate Vaccines (eds. Cruse et al.) ISBN 3805549326, particularly vol. 10: 48-114.
{103} Hermanson (1996) Bioconjugate Techniques ISBN: 0123423368 or 012342335X.
{104} Research Disclosure, 453077 (January 2002)
{105} EP-A-0372501
{106} EP-A-0378881
{107} EP-A-0427347
{108} WO93/17712
{109} WO94/03208
{110} WO98/58668
{111} EP-A-0471177
{112} WO00/56360
{113} WO91/01146
{114} WO00/61761
{115} WO01/72337
{116} Robinson & Torres (1997) Seminars in Immunology 9: 271-283.
{117} Donnelly et al. (1997) Annu Rev Immunol 15: 617-648.
{118} Scott-Taylor & Dalgleish (2000) Expert Opin Investig Drugs 9: 471-480.
{119} Apostolopoulos & Plebanski (2000) Curr Opin Mol Ther 2: 441-447.
{120} Ilan (1999) Curr Opin Mol Ther 1: 116-120.
{121} Dubensky et al. (2000) Mol Med 6: 723-732.
{122} Robinson & Pertmer (2000) Adv Virus Res 55: 1-74.
{123} Donnelly et al. (2000) Am J Respir Crit Care Med 162 (4 Pt 2): S190-193.
{124} Davis (1999) Mt. Sinai J. Med. 66: 84-90.
{125} Current Protocols in Molecular Biology (F. M. Ausubel et al., eds., 1987) Supplement 30.
{126} Smith & Waterman (1981) Adv. Appl. Math. 2: 482-489.

1. A composition comprising two or more of the following gonococcal antigens: (1) OmpA; (2) OmpH; (3) PPIase; (4) ngs41; (5) ngs117; and (6) App.

2. The composition of claim 1, wherein the OmpA protein comprises an amino acid sequence:
(a) having 70% or more identity to SEQ ID : 2; and/or (b) which is a fragment of at least 10 consecutive amino acids of SEQ ID : 2.

3. The composition of claim 1, wherein the OmpH protein comprises an amino acid sequence:
(a) having 70% or more identity to SEQ ID : 3; and/or (b) which is a fragment of at least 10 consecutive amino acids of SEQ ID : 3.

4. The composition of claim 1, wherein the PPIase protein comprises an amino acid sequence:
(a) having 70% or more identity to SEQ ID : 4; and/or (b) which is a fragment of at least 10 consecutive amino acids of SEQ ID : 4.

5. The composition of claim 1, wherein the Ngs41 protein comprises an amino acid sequence:
(a) having 70% or more identity to SEQ ID : 5; and/or (b) which is a fragment of at least 10 consecutive amino acids of SEQ ID : 5.

6. The composition of claim 1, wherein the Ngs117 protein comprises an amino acid sequence:
(a) having 70% or more identity to SEQ ID : 6; and/or (b) which is a fragment of at least 10 consecutive amino acids of SEQ ID : 6.

7. The composition of claim 1, wherein the App protein comprises an amino acid sequence:
(a) having 70% or more identity to SEQ ID : 7; and/or (b) which is a fragment of at least 10 consecutive amino acids of SEQ ID : 7.

8. A hybrid polypeptide of formula NH₂—A—{—X—L—}ₙ—B—COOH, wherein: each X is an amino acid sequence as defined in any one of claims 2 to 7; L is an optional linker amino acid sequence; A is an optional N terminal amino acid sequence; B is an optional C terminal amino acid sequence;
and n is 2, 3, 4, 5, 6, 7, 8, 9, 10, 11, 12, 13 14 or 15.

9. Nucleic acid encoding the hybrid polypeptide of claim 8.

10. A lipidated gonococcal OmpA protein.

11. A lipidated gonococcal PPIase protein.

12. A dimeric gonococcal OmpH protein.

13. A dimeric gonococcal PPIase protein.

14. A gonococcus strain, wherein one or more of the following gonococcal antigens is knocked out:
(1) OmpA; (2) OmpH; (3) PPIase; (4) ngs41; (5) ngs117; and (6) App.
